(19) United States
(12) Patent Application Publication
(10) Pub. No.: US 2019/0060660 A1
(43) Pub. Date: Feb. 28, 2019

(54) STERILIZABLE SKIN CARE DEVICE FOR WRINKLE CARE

(71) Applicant: HEADLINE CO., LTD., Gwangju (KR)

(72) Inventor: Ho Jun LEE, Gwangju (KR)

(21) Appl. No.: 15/578,549

(22) PCT Filed: Oct. 17, 2017
(86) PCT No.: PCT/KR2017/011460

§ 371 Date: Nov. 30, 2017

(30) Foreign Application Priority Data
Aug. 30, 2017 (KR) 10-2017-0109829

(51) Int. Cl. A61N 5/06; A45D 44/00

(52) U.S. Cl. CPC A61N 5/0616; A45D 44/00; A61N 2005/0644; A61N 2005/0626; A61N 2005/0632; A45D 2200/205

(57) ABSTRACT

The skin care beauty equipment capable of sterilization is provided. The skin care beauty equipment capable of sterilization according to the embodiment of the present invention is composed of

a main body that operates by using the charged electric power and stimulates the skin by using a predetermined operation; and
a mounting bracket that operates in either one of a charging mode and a sterilizing mode in accordance with the coupling direction of the above mentioned main body;
wherein, the above mentioned main body is composed of:
a 1st charging section connected to the above mentioned mounting bracket for suppling the electric power from the mounting bracket,
a header section for generating at least one item of galvanic ions, light having a specific wavelength, and vibration to stimulate the above mentioned skin,
and a sensor section for judging whether or not to perform the operation of the above mentioned header section.

### TECHNICAL FIELD

The present invention is related to a skin care beauty equipment capable of sterilization, and to be more specific, it is related to a skin care beauty equipment capable of sterilizing a portion where the skin and the device are in contact with each other.

### BACKGROUND OF THE TECHNOLOGY

The aging process of the skin of human being continues after the age of 20's, and the visual beauty decreases. In particular, in case of the skin of the face which is used to decisively recognize the impression of one person to others and judged the age, a lot of efforts have been made to delay the aging of the skin of the face.

On the other hand, galvanic is a very weak electric current which has the property of pushing between the same poles, ionizing the cosmetics and penetrating into the skin, thereby improving the skin and blurring the melanin pigment such as the spots due to the weak acne; and it has been used as a method of skin beauty that exhibits the effect of improving the wrinkles by the fine stimulation due to the ion emission.

However, for such kind of conventional technologies, the skin care is performed through the contact between the skin and the equipment, but, the function of cleaning or sterilizing is not provided, so, there is a problem that the skin troubles due to various fungi are additionally caused at the time of the skin care.

### DETAILED EXPLANATION OF THE INVENTION

### Technical Tasks

In order to solve the problematic points of the conventional technology as mentioned above, one embodiment of the present invention is to provide a skin care beauty equipment capable of sterilization by maintaining the cleanliness through sterilizing the portion in contact with the skin.

### Technical Solution

According to one aspect of the present invention for the purpose of solving the above mentioned problems, the skin care beauty equipment capable of sterilization is provided. The above mentioned skin care beauty equipment capable of sterilization is composed of a main body that operates by using the charged electric power and stimulates the skin by using a predetermined operation; and

a mounting bracket that operates in either one of a charging mode and a sterilizing mode in accordance with the coupling direction of the above mentioned main body;
wherein, the above mentioned main body is composed of:
a 1st charging section connected to the above mentioned mounting bracket for suppling the electric power from the mounting bracket,
a header section for generating at least one item of galvanic ions, light having a specific wavelength, and vibration to stimulate the above mentioned skin,
and a sensor section for judging whether or not to perform the operation of the above mentioned header section.

The above mentioned mounting bracket is composed of

a coupling form judging section for judging the coupling form of the above mentioned main body and the above mentioned mounting bracket;
a normal coupling judging section for judging whether or not the above mentioned main body is normally coupled to the above mentioned mounting bracket;
a coupling section coupled to the above mentioned main body to supply the above mentioned electric power to the above mentioned main body or to perform the sterilization;
an operation controlling section for selecting either one of the above mentioned charging mode or the above mentioned sterilizing mode according to the above mentioned coupling form, and controlling the operation of the above mentioned coupling section according to the selected mode; and
a setting storage section for storing the setting information about the operation of the above mentioned header section, and at least one mode of the above mentioned charging mode and the above mentioned sterilizing mode;
wherein,
in the case when the above mentioned 1st charging section has not been connected to the above mentioned coupling section, it will be possible operate in the above mentioned charging mode, and in the case when the above mentioned header section has been connected to the coupling section, it will be possible operate in the above mentioned sterilizing mode.

The above mentioned coupling form judging section will be possible to judge the above mentioned coupling form by analyzing whether or not the above mentioned header section or the above mentioned 1st charging section is coupled to the above mentioned coupling section.

The above mentioned normal coupling judging section will be possible to judge whether or not the above mentioned main body is normally coupled to the above mentioned coupling section.

In the case when the above mentioned coupling form analyzed by the coupling form judging section is that the above mentioned header section and above mentioned the coupling section are coupled, the above mentioned operation controlling section will be possible to judge it as the sterilizing mode and performs the sterilizing operation; in the case when the above mentioned coupling form is that the above mentioned 1st charging section and the above mentioned 2nd charging section are coupled, it will be possible to be judged as the above mentioned charging mode, and the charging operation will be possible to be performed.

The above mentioned coupling section can be composed of

a sterilizing section for performing the above mentioned sterilizing operation for the purpose of sterilizing the above mentioned header section; and
and a 2nd charging section for performing the above mentioned charging operation for the purpose of charging the above mentioned main body.

In the case of operating in the above mentioned sterilizing mode, the above mentioned sterilizing section will be possible to sterilize the above mentioned header section by using the UV LED.

In the case of operating in the above mentioned charging mode, the above mentioned 2nd charging section will be possible to charge the above mentioned main body by supplying the above mentioned electric power to the above mentioned 1st charging section.

### Effects of the Invention

The skin care beauty equipment capable of sterilization according to one embodiment of the present invention has the effect of being possible to sterilize a portion in contact with the skin of the user, thereby preventing contamination of the device due to an increase in the time of use.

In addition, the skin care beauty equipment capable of sterilization according to one embodiment of the present invention has the effect of being possible to prevent the skin troubles caused by the bacteria grown on the equipment which may occur due to the use of the equipment through sterilizing the portion in contact with the skin of the user.

In addition, the skin care beauty equipment capable of sterilization according to one embodiment of the present invention has the effect of being possible to prevent the occurrence of the additional sterilization cost by simply providing the sterilization operation in the holder.

### BRIEF EXPLANATION OF THE FIGURES

FIG. 1 is a diagram which has shown (a) a state in which a main body of the skin care beauty equipment capable of sterilization is mounted on a mounting bracket, and (b) the coupling portion of the mounting bracket.

FIG. 2 is a block diagram which has shown the constitution of the skin care beauty equipment capable of sterilization according to an embodiment of the present invention.

FIG. 3 is a block diagram which has shown the constitution of the coupling portion of FIG. 2.

### FORM FOR THE IMPLEMENTATION OF THE INVENTION

Hereinafter, the embodiments of the present invention will be explained in detail with reference to the accompanying figures in a manner that those skilled in the technical field to which the present invention belongs can easily implement. The present invention may be embodied in many different forms and is not limited to the embodiments explained herein. In order to clearly illustrate the present invention, the parts irrelevant to the explanation were omitted, and the same or similar components are denoted by the same reference numerals throughout this whole document of “Detailed Statement”.

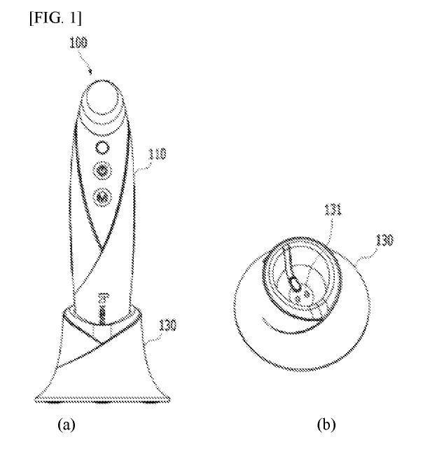

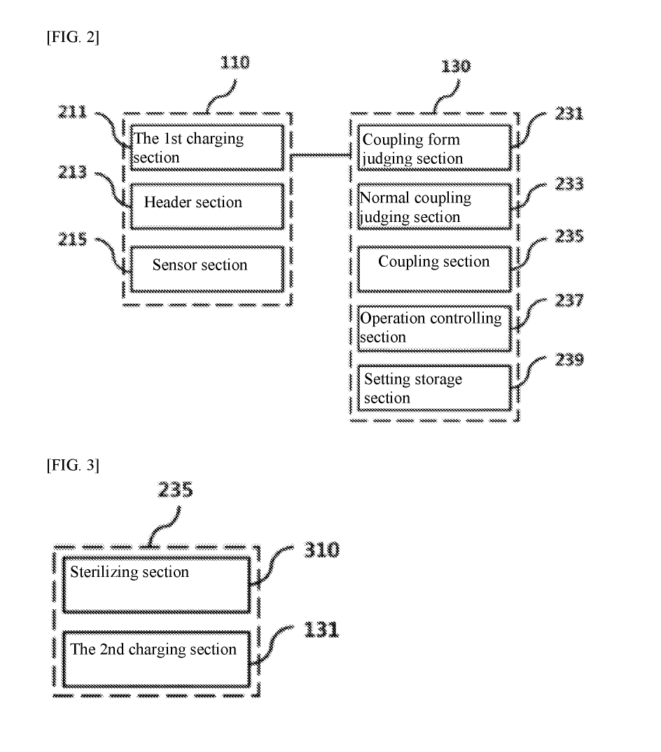

FIG. 1 is a diagram which has shown (a) a state in which a main body of the skin care beauty equipment capable of sterilization is mounted on a mounting bracket, and (b) the coupling portion of the mounting bracket; FIG. 2 is a block diagram which has shown the constitution of the skin care beauty equipment capable of sterilization according to an embodiment of the present invention; FIG. 3 is a block diagram which has shown the constitution of the coupling portion of FIG. 2.

Hereinafter, the skin care beauty equipment capable of sterilization according to one embodiment of the present invention will be explained in more details with reference to FIG. 1 to FIG. 3. However, all the following explanations are only one example of the skin care beauty equipment capable of sterilization of the present invention, and it is self-evident to those skilled in the field that the additions and the modifications of the easily expandable components are also included in the scope of the present invention.

Referring to FIG. 1a and FIG. 1b, the skin care beauty equipment (100) capable of sterilization according to one embodiment of the present invention is formed in a manner that the main body (110) can be coupled to the mounting bracket (130). For the main body (110), by being coupled with the mounting bracket (130), the battery (not shown in the figure) included in the inner side can be charged by using the electric power received from the mounting bracket (130), and it is possible to be operated by using the charged battery.

Here, the main body (110) is composed of a 1st charging section (211) for receiving the electric power from the mounting bracket (130), a header section (213) for performing the skin care operation, and a sensor section (215) for deciding the execution of the header section (213).

The 1st charging section (211) is formed on one side of the main body (110) and receives the electric power from the mounting bracket (130). The 1st charging section (211) may be formed in the form of, for example, a plug; the power supplied from the mounting bracket (130) can be transmitted to the battery formed inside the main body (110) so as to charge the battery.

The header section (213) is formed on the other side of the main body (110) and performs the skin care operation. The header section (213) performs the operation of generating at least one item of galvanic ions, light having a specific wavelength, and vibration, and the skin care can be performed by stimulating the skin through using at least one item of ions, light, and vibration.

The header section (213) may be formed of a variety of materials except for the insulating material in which the electric current does not flow completely; due to the properties of these materials, in the case if the header section (213) touches the skin, the electric current can also move between the skin and the header section (213).

The sensor section (215) measures the electric current to perform the operation of the header section (213). In the case when the user holds the sensor section (215) by hand and contacts the skin with the header section (213), the body of the user, the header section (213) and the sensor section (215) form a kind of electrical circuit. At this time, when the sensor section (215) measures the electric current flowing in the electrical circuit formed by the user and the main body (110), in the case if the electric current can be measured out with the sensor section (215), the main body (110) may perform the operation of generating at least one item of galvanic ions, light having a specific wavelength and vibration, which is a skin care operation from the header section (213).

In addition, in the case when the electric current had not been measured out from the sensor section (215), the main body (110) judges as that currently the user is not using the skin care beauty equipment (100) capable of sterilization, and it is possible to prevent the unnecessary power consumption and the malfunction.

On the other hand, the mounting bracket (130) may be coupled to the main body (110), and may operate in a charging mode or a sterilizing mode according to the direction of the main body (110). For this reason, the mounting bracket (130) may be formed with a coupling form judging section (231), a normal coupling judging section (233), a coupling section (235), an operation controlling section (237), and a setting storage section (239).

The coupling form judging section (231) judges the form in which the main body (110) is coupled to the mounting bracket (130). The coupling form judging section (231) judges in which direction the main body (110) is coupled to the mounting bracket (130). In other words, the main body (110) is provided with the 1st charging section (211) on one side and the header section (213) formed on the other side.

That is to say, the coupling form judging section (231) is possible to judge whether the 1st charging section (211) of the main body (110) is coupled to the mounting bracket (130) or whether the header section (213) is coupled to the mounting bracket (130).

The coupling form judging section (231) may further include an optical sensor, a magnetic field sensor, an electric current sensor, or the like to judge the coupling direction of the main body (110); as an example, in the case when an electric current sensor is included, by forming the electric current sensor at a position in contact with the header section (213) but not in contact with the 1st charging section (211), it will be possible to judge the coupling form with the flowing electric current in the case if the header section (213) is coupled.

The normal coupling judging section (233) judges whether the main body (110) is normally coupled to the mounting bracket (130). When it is judged with the coupling form judging section (231) whether the main body (110) coupled with the mounting bracket (130) is the 1st charging section (211) or the header section (213), the normal coupling judging section (233) judges whether the coupling of the main body (110) is normal or not.

The normal coupling judging section (233) may judge whether the main body (110) has a predetermined center of gravity according to a direction in which the main body (110) is coupled through utilizing the weight sensor as one example, and by this way, it is possible to judge whether or not the coupling criterion is satisfied or not. In other words, in the case when the predetermined center of gravity is different from the measured center of gravity, the normal coupling judging section (233) judges that the main body (110) is not normally coupled with the mounting bracket (130), and it may also be possible to provide an alert to the user requesting extra coupling.

The coupling section (235) is formed in a way of coupling the main body (110) and supplying the electric power to the coupled main body (110) or perform the sterilization. The coupling section (235) is formed for the purpose of coupling the main body (110) in the mounting bracket (130) and perform a predetermined operation in the main body (110). In other words, when the main body (110) is coupled to the coupling section (235), the coupling form judging section (231) and the normal coupling judging section (233) can be provided so as to judge whether the form of the main body (110) coupled to the coupling section (235) is a normal coupling or not.

On the other hand, the coupling section (235) may further include a sterilizing section (310) for the purpose of performing the predetermined operations in the main body (110) and a 2nd charging section (131).

The sterilizing section (310) is provided to sterilize the main body (110). The sterilizing section (310) is formed inside the coupling section (235) for the purpose of sterilizing the header section (213) in the case when the header section (213) of the main body (110) is coupled with the coupling section (235). At this time, the sterilizing section (310) may be formed of at least one UV LED for the purpose of sterilizing a wide area of the header section (213).

As one example, the sterilizing section (310) may be provided with at least one UV LED on the bottom part of the coupling section (235) for the purpose of sterilizing both the surface where the header section (213) is in contact with the skin and the adjacent surface of the header section (213), and a plurality of UV LEDs are formed on the side surface of the coupling section (235), so that the entire area of the header section (213), which is exposed to the outside with the possibility of being contaminated, can be sterilized.

In addition, the sterilizing section (310) may also be formed with 1 UV LED on the side surface of the coupling section (235), so that it is also possible to sterilize the adjacent surface of the header section (213) while rotating along the trajectory previously formed on the adjacent surface of the coupling section (235). Such kind of constitution is only one illustrative example of the present invention, and the present invention is not necessarily limited to this example.

On the other hand, the 2nd charging section (131) is formed for the purpose of supplying the electric power to the main body (110) to charge the main body (110). The 2nd charging section (131) is preferably formed in a manner of being additionally coupled with the 1st charging section (211) formed on one side of the main body (110). The 2nd charging section (131) can be formed in a manner of being in contact with the 1st charging section (211) to transmit the electric power provided from the other side to the 1st charging section (211), so that the 1st charging section (211) can charge the main body (110).

At this time, the 2nd charging section (131) may be preferably formed on the bottom surface of the coupling part 235, however, the present invention is not limited to this constitution, and it can also be formed (211) at a position where the contact with the 1st charging section (211) is possible along the direction of the 1st charging section (211).

The operation controlling section (237) selects a mode according to the coupling form, and controls the operation of the coupling section (235) according to the selected mode. The operation controlling section (237) transmits the direction of the main body (110) coupled with the coupling section (235) from the coupling form judging section (231). At this time, when it is judged as that the 1st charging section (211) was coupled to the coupling section (235), the operation controlling section (237) is possible to control so as to switch the operation mode of the mounting bracket (130) to the charging mode, operate the 2nd charging section (131) and perform the charging operation during which the electric power is transmitted to the 1st charging section (211).

In addition, when it is judged as that the header section (213) was coupled to the coupling section (235), the operation controlling section (237) is possible to control so as to switch the operating mode of the mounting bracket (130) to the sterilizing mode, operate the sterilizing section (310), and perform the sterilizing operation for implementing the sterilization of the header section (213).

The setting storage section (239) stores various settings for the purpose of judging the operations of the main body (110) and the mounting bracket (130). The setting storage section (239) receives the operation-related settings from a user, stores the input settings, and transmits the settings to the operation controlling section (237) and the main body (110).

In other word, when the user inputs a setting for the purpose of maintaining the operation of the main body (110) for 5 a period of minutes, the setting storage section (239) transmits the information to the main body (110), in this way, even if the electric current measurement is finished via the sensor section (215), it will be possible to maintain the operation for a period of 5 minutes according to the setting of the user.

In addition, the setting storage section (239) may also store the sterilization time and the cycle-related information of the sterilizing section (310), and may prevent the overcharge by controlling the charging time and the charging electric current of the 2nd charging section (131).

In one embodiment of the present invention, such setting storage section (239) is formed on the mounting bracket (130) for the convenience of power consumption and communication, and it has been explained in a manner wherein in the case when the mounting bracket (130) and the main body (110) are coupled, the information can be transmitted to the main body (110), however, the present invention is not limited to this explanation, and it may also be provided on the main body (110) according to the needs of the user or the necessity.

In the above, the present invention has been explained with reference to the exemplary embodiment, but, the idea of the present invention is not limited to the embodiment presented in this Detailed Statement, those skilled in this industry who understand the idea of the present invention can easily propose other embodiments through the supplementation, the changes, the deletion, the addition or the like of the constituent elements within the scope of the same idea, and these embodiments should also be included within the scope of the idea of the present invention.

1. The skin care beauty equipment capable of sterilization which is composed of
a main body that operates by using the charged electric power and stimulates the skin by using a predetermined operation; and
a mounting bracket that operates in either one of a charging mode and a sterilizing mode in accordance with the coupling direction of the above mentioned main body;
wherein, the above mentioned main body is composed of:
a 1st charging section connected to the above mentioned mounting bracket for suppling the electric power from the mounting bracket,
a header section for generating at least one item of galvanic ions, light having a specific wavelength, and vibration to stimulate the above mentioned skin,
and a sensor section for judging whether or not to perform the operation of the above mentioned header section.

2. The skin care beauty equipment capable of sterilization as described in the claim 1, of which the above mentioned mounting bracket is composed of
a coupling form judging section for judging the coupling form of the above mentioned main body and the above mentioned mounting bracket;
a normal coupling judging section for judging whether or not the above mentioned main body is normally coupled to the above mentioned mounting bracket;
a coupling section coupled to the above mentioned main body to supply the above mentioned electric power to the above mentioned main body or to perform the sterilization;
an operation controlling section for selecting either one of the above mentioned charging mode or the above mentioned sterilizing mode according to the above mentioned coupling form, and controlling the operation of the above mentioned coupling section according to the selected mode; and
a setting storage section for storing the setting information about the operation of the above mentioned header section, and at least one mode of the above mentioned charging mode and the above mentioned sterilizing mode;
wherein,
in the case when the above mentioned 1st charging section has not been connected to the above mentioned coupling section, it will operate in the above mentioned charging mode, and in the case when the above mentioned header section has been connected to the coupling section, it will operate in the above mentioned sterilizing mode.

3. The skin care beauty equipment capable of sterilization as described in the claim 2, wherein the above mentioned coupling form judging section judges the above mentioned coupling form by analyzing whether or not the above mentioned header section or the above mentioned 1st charging section is coupled to the above mentioned coupling section.

4. The skin care beauty equipment capable of sterilization as described in the claim 3, wherein the above mentioned normal coupling judging section judges whether or not the above mentioned main body is normally coupled to the above mentioned coupling section.

5. The skin care beauty equipment capable of sterilization as described in the claim 4, wherein in the case when the above mentioned coupling form analyzed by the coupling form judging section is that the above mentioned header section and above mentioned the coupling section are coupled, the above mentioned operation controlling section judges it as the sterilizing mode and performs the sterilizing operation; in the case when the above mentioned coupling form is that the above mentioned 1st charging section and the above mentioned 2nd charging section are coupled, it will be judged as the above mentioned charging mode, and the charging operation will be performed.

6. The skin care beauty equipment capable of sterilization as described in the claim 5, wherein the above mentioned coupling section is composed of
a sterilizing section for performing the above mentioned sterilizing operation for the purpose of sterilizing the above mentioned header section; and
and a 2nd charging section for performing the above mentioned charging operation for the purpose of charging the above mentioned main body.

7. The skin care beauty equipment capable of sterilization as described in the claim 6, wherein in the case of operating in the above mentioned sterilizing mode, the above mentioned sterilizing section will sterilize the above mentioned header section by using the UV LED.

8. The skin care beauty equipment capable of sterilization as described in the claim 7, wherein in the case of operating in the above mentioned charging mode, the above mentioned 2nd charging section will charge the above mentioned main body by supplying the above mentioned electric power to the above mentioned 1st charging section.
